(19) United States
(12) Patent Application Publication
(10) Pub. No.: US 2010/0130837 A1
(43) Pub. Date: May 27, 2010

(54) Modular ingestible capsule

(75) Inventor: Laura A. Matott, East Aurora, NY (US)

Correspondence Address: PHILLIPS LYTLE LLP;INTELLECTUAL PROPERTY GROUP, 3400 HSBC CENTER, BUFFALO, NY, 14203-3509, US

(73) Assignee: The Smart Pill Corporation

(21) Appl. No.: 12/313,856

(22) Filed: Nov. 25, 2008

(51) Int. Cl. A61B 5/07

(52) U.S. Cl. 600/302

(57) ABSTRACT

A modular ingestible capsule (15) comprising a capsule body (16) having an outer shell (21), a power supply (22), a transmitter (23) connected with the power supply, an antenna (24) connected with the transmitter, an activator (25) configured to activate the power supply, and an electrical coupling element (26) connected with the transmitter, a first modular sensing component (18) configured and arranged to connect to the capsule body and having an outer shell (28) configured and arranged to attach to the shell of the capsule body, a sensor (29) for sensing a parameter of an ingestible tract, and an electrical coupling element (32) connected with the sensor and configured and arranged to engage the electrical coupling element of the capsule body, whereby the first modular sensing component may be readily mechanically and electrically attached to the capsule body prior to ingestion of capsule by subject.

### TECHNICAL FIELD

The present invention relates generally to ingestible capsules and, more particularly, to an ingestible capsule having interchangeable sensing components.

### BACKGROUND ART

Ingestible capsules are well-known in the prior art. Such capsules are generally small pill-like devices that can be ingested or swallowed by a patient. It is known that such capsules may include one or more sensors for determining physiological parameters of the gastrointestinal tract, such as sensors for detecting temperature, pH and pressure.

It is also known that certain physiological parameters may be associated with regions of the gastrointestinal tract. For example, a 1988 article entitled “Measurement of Gastrointestinal pH Profiles in Normal Ambulant Human Subjects” discloses pH measurements recorded by a capsule passing through the gastrointestinal tract. It is known that pH has been correlated with transitions from the stomach to the small bowel (gastric emptying) and from the distal small bowel to the colon (ileo-caecal junction).

U.S. Patent Publication Number US2007/0118012 discloses an imaging device having two optical heads, and discloses that domes may be placed over the optical heads and into abutment with a connecting sleeve so that the connecting sleeve and the domes form a closed housing that defines the boundary surface of the in-vivo device. International Publication Number WO 2006/0077535 discloses a medicament dispensing capsule in which individual reservoirs may be provided in respective modules which are interlocking and connectable. International Publication Number WO 2006/0070374 discloses a system and method for assembling a swallowable sensing device, including attaching a first piece of a shell to a second piece of a shell, where the attachment may include, for example, screwing the first piece to the second piece, welding or gluing the first piece to the second piece, snapping the first piece to the second piece or applying laser energy to a pigment in the first piece.

### DISCLOSURE OF THE INVENTION

With parenthetical reference to corresponding parts, portions or surfaces of the disclosed embodiment, merely for the purposes of illustration and not by way of limitation, the present invention provides a modular ingestible capsule (15) comprising a capsule body (16) having an outer shell (21), a power supply (22), a transmitter (23) connected with the power supply, an antenna (24) connected with the transmitter, an activator (25) configured to activate the power supply, and an electrical coupling element (26) connected with the transmitter, a first modular sensing component (18) configured and arranged to connect to the capsule body and having an outer shell (28) configured and arranged to attach to the shell of the capsule body, a sensor (29) for sensing a parameter of an ingestible tract, and an electrical coupling element (32) connected with the sensor and configured and arranged to engage the electrical coupling element of the capsule body, whereby the first modular sensing component may be readily mechanically and electrically attached to the capsule body prior to ingestion of the capsule by subject.

The sensor may be selected of a group consisting of a pH sensor, a pressure sensor and a temperature sensor. The power supply may comprise a battery. The transmitter may be configured to transmit data to a remote receiver. The capsule body my further comprise a temperature sensor (27). The electrical coupling element of the capsule body may comprise a plug and the electrical coupling element of the modular sensing component may comprise a receptacle configured to receive the plug. The modular sensing component may further comprise a processor (31) connected with the sensor. The processor may be programmed to control the sampling rate of the sensor. The processor may be programmed to control the transmission burst duration and the rate of transmission bursts. The sensor may be an analog sensor (30) that provides an output voltage in response to stimuli. The modular sensing component may further comprise an analog-digital converter (41) configured to convert an analog signal from the sensor to a digital signal. The capsule body may further comprise a processor (74) connected with the electrical coupling element (75) of the body and the modular sensing component may further comprise an ID tag recognizable to the processor.

The capsule may further comprise a second modular sensing component (20) configured and arranged to connect to the capsule body and having an outer shell (33) configured and arranged to attach to the shell of the capsule body, a sensor (30) for sensing a parameter of an ingestible tract, and an electrical coupling element (35) connected with the sensor and configured and arranged to engage the electrical coupling element of the capsule body, wherein the second modular sensing component may be interchangeably attached mechanically and electrically to the capsule body with the first modular sensing component.

The capsule body (50) may comprise a second electrical coupling element (58) connected with the transmitter and the capsule may further comprise a second modular sensing component (20) configured and arranged to connect to the capsule body and having an outer shell (33) configured and arranged to attach to the shell (52) of the capsule body, a sensor (30) for sensing a parameter of the ingestible tract, and an electrical coupling element (35) connected with the sensor and configured and arranged to engage the second electrical coupling element of the capsule body, whereby the second modular sensing component may be mechanically and electrically attached to the capsule body prior to ingestion of the capsule by a subject.

The sensor of the first modular sensing component may be different from the sensor of the second modular sensing component, and the sensor of the first modular sensor component may be a pH sensor (30) and the sensor of the second modular sensing component may be a pressure sensor (29). The sensor of the first modular sensing component may be the same as the sensor of the second modular sensing component, and the sensors may be pressure sensors. The transmitter may be connected directly to the power supply and the processor may communicate with the sensor bi-directionally. The electrical coupling element of the body may be connected to the power supply, and the electrical coupling element of the body may be connected directly to the power supply and the transmitter.

In another aspect, the invention provides a method of measuring parameters of the gastrointestinal tract of a subject comprising the steps of providing a capsule body having an outer shell, a power supply, a transmitter connected to the power supply, an antenna connected with the transmitter, an activator configured to activate the power supply and an electrical coupling element connected with the transmitter, providing a first modular sensing component configured and arranged to connect to the capsule body and having an outer shell configured and arranged to attach to the shell of the capsule body, a sensor for sensing a parameter of an ingestible tract, and an electrical coupling element connected with the sensor and configured and arranged to engage the electrical coupling element of the capsule body, providing a second modular sensing component configured and arranged to connect to the capsule body and having an outer shell configured and arranged to attach to the shell of the capsule body, a sensor for sensing a parameter of an ingestible tract, and an electrical coupling element connected with the sensor and configured and arranged to engage the electrical coupling element of the capsule body, attaching one of the first or second modular sensing components to the capsule body, having a subject ingest the capsule, recording measurements from the sensor of the attached modular sensing component as the capsule passes through a gastrointestinal tract of the subject, and transmitting the measurements from a transmitter to a receiver located outside of the gastrointestinal tract of the subject.

Accordingly, an object is to provide a modular capsule system in which different sensing components may be interchangeably used with a standard base component.

Another object is to provide a method for customizing an ingestible capsule using interchangeable sensing components.

These and other objects and advantages will become apparent from the foregoing and ongoing written specification, the drawings, and the claims.

### BRIEF DESCRIPTION OF THE DRAWINGS

FIG. 1 is a schematic of a first embodiment of the modular ingestible capsule system.

FIG. 2 is a front plane view of a first embodiment of the modular ingestible capsule.

FIG. 3 is a longitudinal vertical sectional view of the modular capsule shown in FIG. 2, taken generally on line A-A of FIG. 2.

FIG. 4 is an exploded view of the modular capsule shown in FIG. 2.

FIG. 5 is a longitudinal vertical sectional view of the modular capsule shown in FIG. 4, taken generally on line B-B of FIG. 4.

FIG. 6 is a top plan view of the pressure modular cap shown in FIG. 1.

FIG. 7 is a transverse vertical sectional view of the modular cap shown in FIG. 6, taken generally on line C-C of FIG. 6.

FIG. 8 is a top plan view of the pH modular cap shown in FIG. 1.

FIG. 9 is a longitudinal vertical sectional view of the modular cap shown in FIG. 8, taken generally on line D-D of FIG. 7.

FIG. 10 is a front plan view of the pH and pressure modular cap shown in FIG. 1.

FIG. 11 is a transverse horizontal sectional view of the modular cap shown in FIG. 10, taken generally on line E-E of FIG. 10.

FIG. 12 is a transverse horizontal sectional view of the modular cap shown in FIG. 10, taken generally on line F-F of FIG. 10.

FIG. 13 is an exploded view of a second embodiment of the modular ingestible capsule.

FIG. 14 is a longitudinal vertical sectional view of the modular capsule shown in FIG. 13, taken generally on line G-G of FIG. 13.

FIG. 15 is a diagram of electrical connections for the modular capsule shown in FIG. 5.

FIG. 16 is an exploded view of a third embodiment of the modular ingestible capsule.

FIG. 17 is a longitudinal vertical sectional view of the modular capsule shown in FIG. 16, taken generally on line H-H of FIG. 16.

FIG. 18 is a diagram of electrical connections for the modular capsule shown in FIG. 17.

### DESCRIPTION OF PREFERRED EMBODIMENTS

At the outset, it should be clearly understood that like reference numerals are intended to identify the same structural elements, portions or surfaces consistently throughout the several drawing figures, as such elements, portions or surfaces may be further described or explained by the entire written specification, of which this detailed description is an integral part. Unless otherwise indicated, the drawings are intended to be read (e.g., cross-hatching, arrangement of parts, proportion, degree, etc.) together with the specification, and are to be considered a portion of the entire written description of this invention. As used in the following description, the terms “horizontal”, “vertical”, “left”, “right”, “up” and “down”, as well as adjectival and adverbial derivatives thereof (e.g., “horizontally”, “rightwardly”, “upwardly” etc.), simply refer to the orientation of the illustrated structure as the particular drawing figure faces the reader. Similarly, the terms “inwardly” and “outwardly” generally refer to the orientation of a surface relative to its axis of elongation, or axis of rotation, as appropriate.

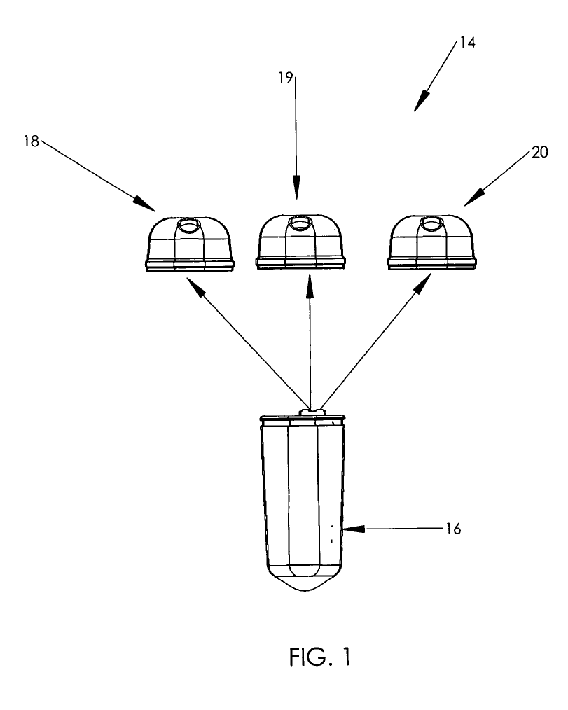

Referring now to the drawings and, more particularly, to FIG. 1 thereof, this invention provides a modular ingestible capsule system for evaluating the gastrointestinal tract of a subject, of which a first embodiment is generally indicated at 14. As shown in FIG. 1, system 14 generally includes a standard capsule body 16 that is adapted to be connected with multiple different but interchangeable modular caps 18, 19 and 20. Body 16 houses certain standard electronics for the capsule and each of caps 18, 19 and 20 are attachable to body 16 and include different sensors or combinations of sensors for sensing parameters of the gastrointestinal tract of a subject. Body 16 and caps 18, 19 and 20 are designed such that the capsule user can choose the cap having the sensor or sensor combination desired at that time and can plug and attach the cap to the capsule body to provide the desired capsule just prior to having the patient ingest the capsule. Thus, system 14 allows the user to selectively customize the ingestible capsule using interchangeable sensing components. Multiple different modular components may be interchangeably used with standard base component 16. While this embodiment describes modular components having pH and pressure sensors, caps having other types of sensors may be used, such as temperature sensors, blood sensors, and imaging sensors.

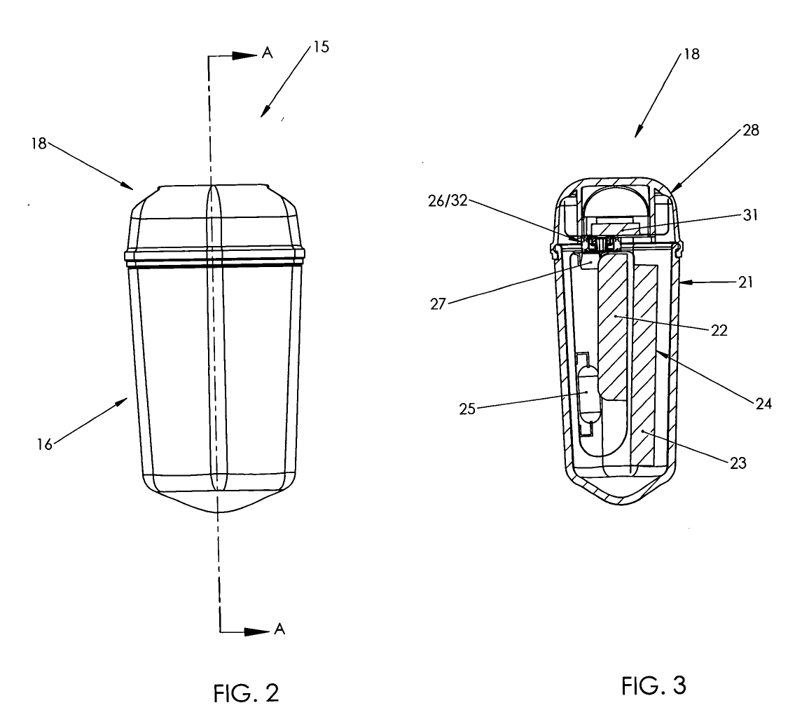

FIG. 2 shows modular cap 18 connected to capsule body 16. As shown, connected they form an elongated generally ellipsoid-shaped device 15, somewhat resembling a medicament capsule. Capsule 15 is adapted to be ingested or otherwise positioned within a tract to sense pressure, pH and/or temperature within the tract and to transmit such readings. The capsule is generally provided with an outer surface or shell to facilitate easy swallowing of the capsule. In the preferred embodiment, capsule 15 is an autonomous swallowable capsule and is self-contained. Thus, capsule 15 does not require any wires or cables to, for example, receive power or transmit information. The pH, pressure and/or temperature data are transmitted from within the GI tract to a remote data receiver.

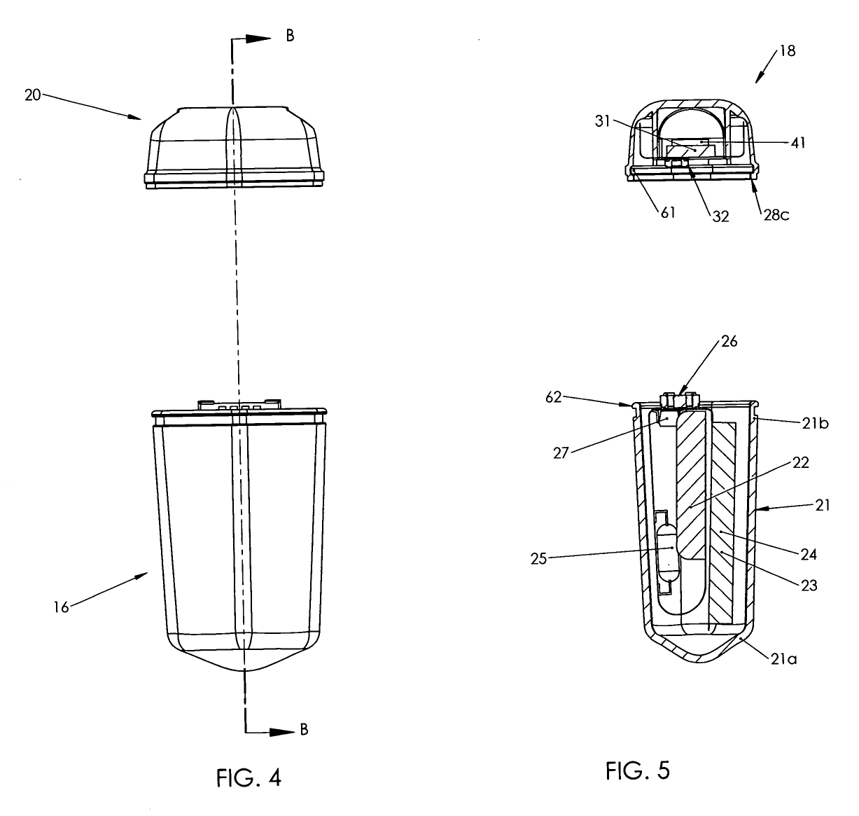

As shown in FIGS. 2-5, standard capsule body 16 generally includes a power supply 22, a transmitter 23, an antenna 24, an activation switch 25 and a temperature sensor 27 housed in a hard shell or casing 21. One end 21 a of casing 21 is rounded and the other end terminates in an annular rim 21b and includes an electrical receptacle 26 adapted to receive a corresponding electrical plug. Power supply 22 and transmitter 23 are connected to each other and to electrical receptacle 26.

In this embodiment, power supply 22 is a lithium battery, although it is contemplated that other batteries may be used, such as a silver-oxide battery. Power supply 22 is adapted to power the electrical components of capsule 15 when in the gastrointestinal tract of a subject.

To maximize its operation life, battery 22 is activated just prior to ingestion by way of a magnetic activation switch 25 adapted to turn the capsule on and off. In this embodiment, activation switch 25 is a circuit operating between battery 22 and the electrical components that selectively powers on and off the electronic components by way of a magnetic sensor which responds selectively to the presence, absence and/or polarity of a magnetic field. A number of conventional switches may be used. For example, an “active” reed switch system may be used, in which an external magnetic field actively holds a reed switch so that the circuit remains open. When the ingestible capsule is removed from the magnetic field, the reed switch closes the circuit, thereby activating the capsule. An alternative method is to use a passive reed switch and a magnetizable bias magnet asymmetric design manipulated by an external magnet. The circuitry of the capsule is selectively switched on and off depending on the magnetic state of the bias magnet, which determines the reed switch on/off state. The magnetic activation and deactivation circuit disclosed in U.S. patent application Ser. No. 11/899,316 entitled “Magnetic Activation and Deactivation Circuit and System,” the entire disclosure of which is incorporated herein by reference, may also be used in this embodiment.

In this embodiment, transmitter 23 is a radio frequency (RF) transmitter that transmits measurements from capsule 15 when it is in the gastrointestinal tract of a subject to a remote receiver. Transmitter 23 transmits measurements at about 434 MHz. A portable data receiver worn by the subject receives and stores the measurements transmitted by transmitter 23 for later download through a docking station to a Windows PC compatible computer, such as a conventional laptop or a desktop. Antenna 24 amplifies the transmit power of transmitter 23 so that it can be received by the remote receiver.

In this embodiment, body 16 also includes a temperature sensor 27 communicating with power supply 22 and electrical receptacle 26. This temperature sensor may be used to compensate or provide a baseline relative to sensors in the modular cap that is connected to base 16.

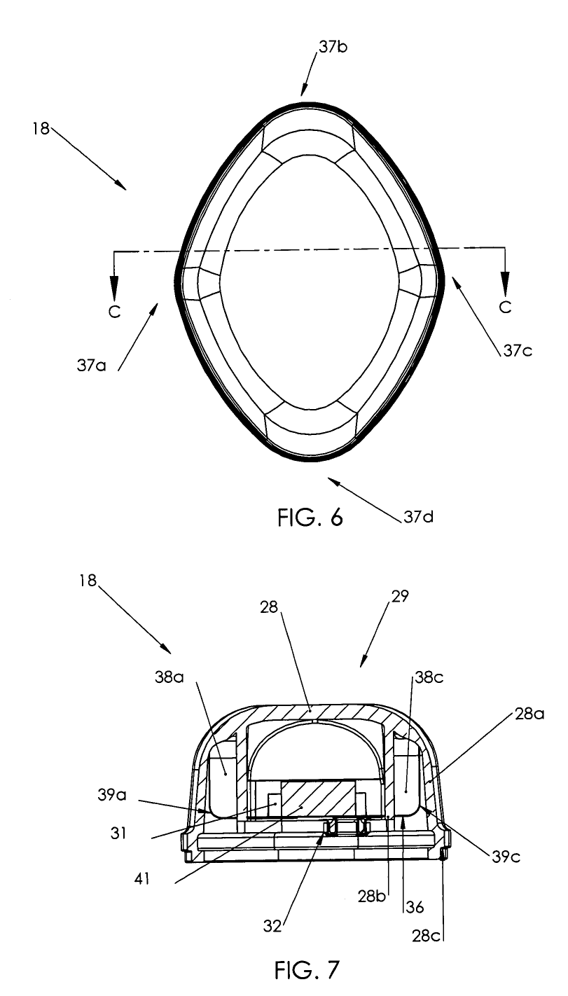

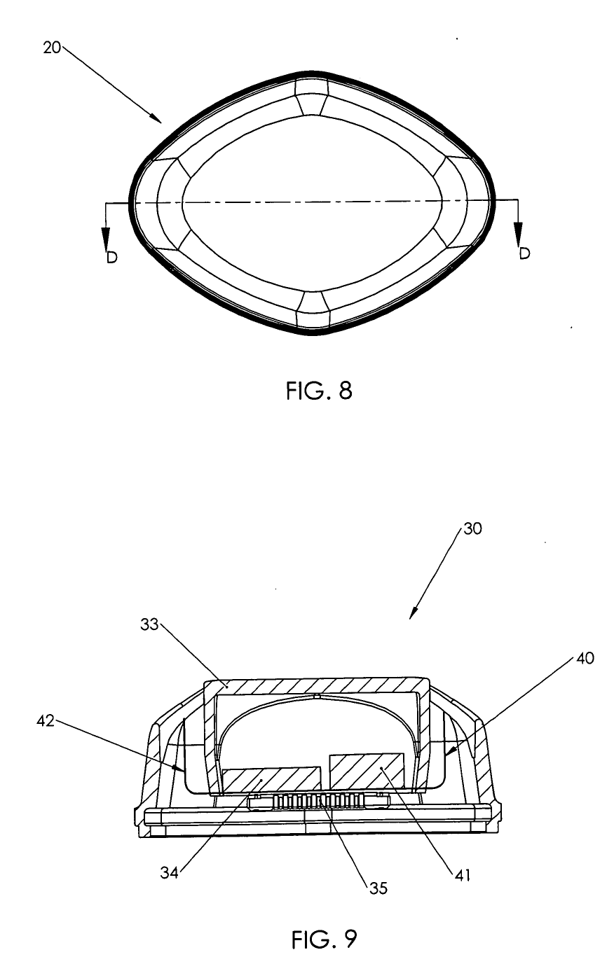

Caps 18, 19 and 20 may be interchangeably used with body 20 to form an ingestible capsule. Cap 18 is adapted to be used with body 16 if the user desires to sense pressure within the gastrointestinal tract of the subject with capsule 15. As shown in FIG. 7, cap 18 has an outer shell 28 that houses a processor 31 and pressure sensor assembly 29. As shown, shell 28 has a top rounded end and terminates at an annular rim 28c.

Pressure sensor assembly 29 includes a chamber 38 between an inner wall 28b and a flexible membrane 28a of shell 28. Chamber 38 is filled with a fluid. A rigid PCB arm 36 extends into the chamber and supports a conventional piezoelectric bridge 39. As fluid presses against bridge 39, it creates an electrical signal which corresponds to the pressure of fluid in chamber 38. The fluid is a non-compressible medium that transfers a force onto the sensing mechanism for sensor 39. In this embodiment, the fluid used is a dielectric gel. Alternatively, it is contemplated that other fluids, such as mineral oil, may be used or an inert gas may be used instead of a fluid. Thus, pressure sensor 39 is operatively arranged to sense pressure within chamber 38. An analog to digital converter 41 is provided to convert the analog signal from sensor 39 to a digital signal.

As shown in FIGS. 6-7, multiple chambers 38a, 38c with multiple pressure sensors 39a, 39c may be included on capsule 18. In this embodiment, cap 18 is somewhat elliptical, and includes four chambers and four pressure sensors 39 located generally at the opposed corners on the major axis 37b and 37d and minor axis 37a and 37c of cap 18.

Pressure sensor assembly 29 is connected to and communicates with micro-processor 31. Processor 31 controls the sampling rate of sensor 29 and is also connected through plug 32 and port 26 to transmitter 23 to control the RF transmission frequency and the information and data being transmitted. Processor 31 may also process signals received from sensor 29 and temperature sensor 27 and may provide other command or control signals to capsule components. The term processor as used herein refers to any data processor. Some examples of processors are microprocessors, microcontrollers, CPUs, PICs, PLCs, PCs or microcomputers. The processor described above is for purposes of example only. Thus, the term processor is to be interpreted expansively.

Cap 20 is adapted to be used with body 16 if the user desires to sense pH within the gastrointestinal tract of the subject with capsule 15. As shown in FIG. 9, cap 20 has an outer shell 33 that houses a processor 34, an analog to digital converter 41, and a pH sensor assembly 30. In this embodiment, pH sensor assembly 30 comprises a conventional ISFET type pH sensor 40 on one side with a pH reference electrode 42 on the other. ISFET stands for ion-selective field effect transistor and the sensor is derived from MOSFET technology (metal oxide screen field effect transistor). A current between a source and a drain is controlled by a gate voltage. The gate is composed of a special chemical layer which is sensitive to free hydrogen ions (pH). Versions of this layer have been developed using aluminum oxide, silicon nitride and titanium oxide. Free hydrogen ions influence the voltage between the gate and the source. The effect on the drain current is based solely on electrostatic effects, so the hydrogen ions do not need to migrate through the pH sensitive layer. This allows equilibrium, and thus pH measurement, to be achieved in a matter of seconds. The sensor is an entirely solid state sensor, unlike glass bulb sensors which require a bulb filled with buffer solution. Only the gate surface is exposed to the sample. An analog to digital converter 41 is provided to convert the analog signal from sensor 40 to a digital signal. The pH ISFET sensor 40 and pH reference electrode 42 extend from the shell 33 in protective channels, such that the sensors are exposed to the medium of the gastrointestinal tract but are protected from breaking or causing damage.

Micro-processor 34 is connected to and communicates with pH sensor 30. Processor 34 controls the sampling rate of sensor 30 and is also connected through plug 35 and port 26 to transmitter 23 to control the RF transmission frequency and the information and data being transmitted. Processor 34 may also process signals received from sensor 30 and temperature sensor 27 and may provide other command or control signals to capsule components.

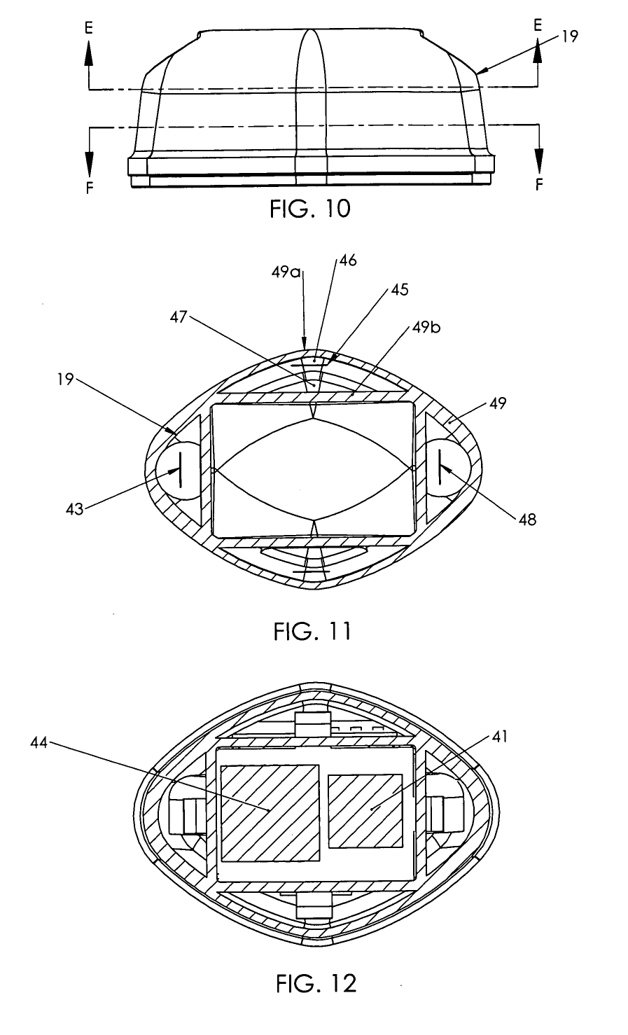

Cap 19 is adapted to be used with body 16 if the user desires to sense both pH and pressure within the gastrointestinal tract of the subject with capsule 15. As shown in FIGS. 10-12, cap 19 has an outer shell 49 that houses a processor 44, an analog to digital converter 44, a pH sensor 48, a pH reference electrode 43, and a pressure sensor assembly 46. As shown, pH sensor 48 and reference electrode 43 are similar to the pH sensor assembly 30 of cap 20. Pressure sensor assembly 46 is similar to pressure sensor assembly 29 of cap 19, with a flexible membrane portion 49a of shell 49 and inner wall 49b of shell 49 defining a chamber 47 containing a piezoelectric bridge 45 operatively arranged to sense pressure within chamber 47. Again, since the output from the sensors is an analog signal, an analog to digital converter 41 is provided to convert the signal from sensor 45 and 48 to a digital signal. Micro-processor 44 is connected to and communicates with pH sensor 48 and pressure sensor 46. Processor 44 controls the sampling rate of sensors 48 and 46 and is also connected through a plug (not shown) and port 26 to transmitter 23 to control the RF transmission frequency and the information and data being transmitted from body 16. Processor 44 may also process signals received from sensors 48 and 46 and temperature sensor 27 and may provide other command or control signals to capsule components.

After activation and ingestion, capsule 15 senses and transmits measurements for at least 120 hours after activation. In the preferred embodiment, the range and accuracy of the sensors are generally 1 to 9.0 pH units with an accuracy of ±0.5 pH units, 0 to 350 mmHg with an accuracy of ±5 mmHg, and 25° to 49° C. with an accuracy of ±1° C.

Caps 18, 19, 20 and capsule body 16 have both mechanical connecting elements and electrical connecting elements. As shown in FIGS. 5 and 7, the shell of the subject modular cap attaches to the shell 21 of body 16 in this embodiment by a snap connection. The bottom peripheral rim 28c of the subject cap includes an annular cavity 61. A corresponding annular protrusion 62 is provided in the top rim 21b of shell 21 of body 20. Annular cavity 61 in the subject cap is configured to receive protrusion 62 of the body. The body and subject cap are thereby pressed together until angular protrusion 62 snaps into corresponding cavity 61, thereby holding the subject cap and body 16 together. However, it is contemplated that other types of connections may be used to attach the subject cap to body 16. For example, shell 28 and shell 21 may include corresponding screw threads with matching grooves along a flange on their outer rims so that the two ends of the shells may be screwed together. Alternatively, the rims of shells 28 and 21 may be attached by gluing or bonding the two parts together or the shells may be threaded and twisted relative to one another to provide a connection.

The electrical connection between the subject cap and body 16 is provided by an I/O connector having an electrical connecting input or plug 32, 35 on the subject cap and an electrical output connection, port or receptacle 26 in body 16. Port 26 is adapted to receive either plug 32 or plug 35 and to electrically connect the subject components of the selected cap and capsule body 16. To form the capsule, the user selects the desired cap, aligns the plug in the subject cap with receptacle 26 of body 16, and then presses the cap and body together until they snap in place, by which an electrical and mechanical connection is formed between them. Thus, the subject cap and body 16 may be easily or readily connected together by the user. The body and subject cap may be connected by hand, and may also be releasably connected such that the subject cap and body may be easily or readily detached from each other.

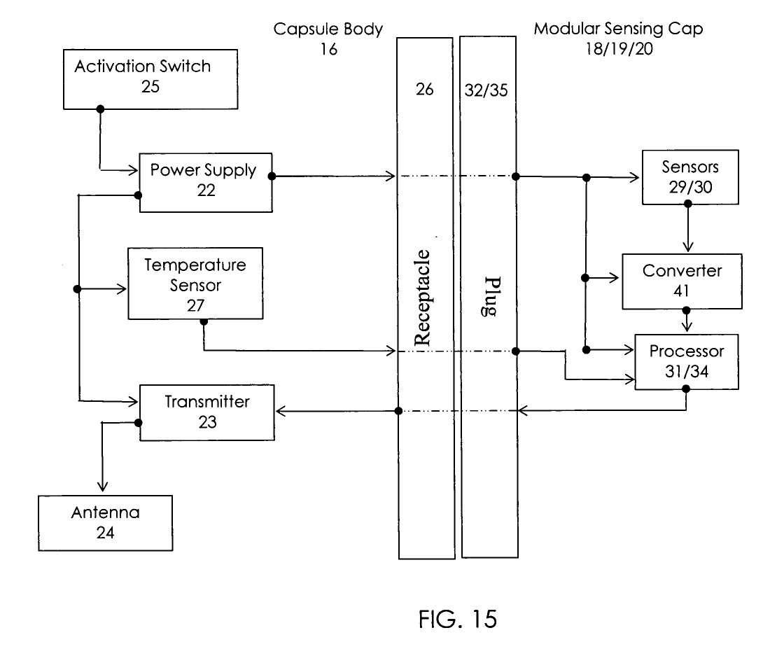

As shown in FIG. 15, power supply 22 is connected to transmitter 23 and temperature sensor 27 in capsule body 16 and is connected through port 26 and plug 32/35 to the subject sensors 29/30, subject processor 31/34 and converter 41. Power supply 22 is, as described above, activated by switch 25. The subject sensors 29/30 are connected through analog to digital converter 41 to the subject processor 31/34. Processor 31/34 is also connected through plug 32/35 and port 26 to transmitter 23. Transmitter 23 is in turn connected to antenna 24.

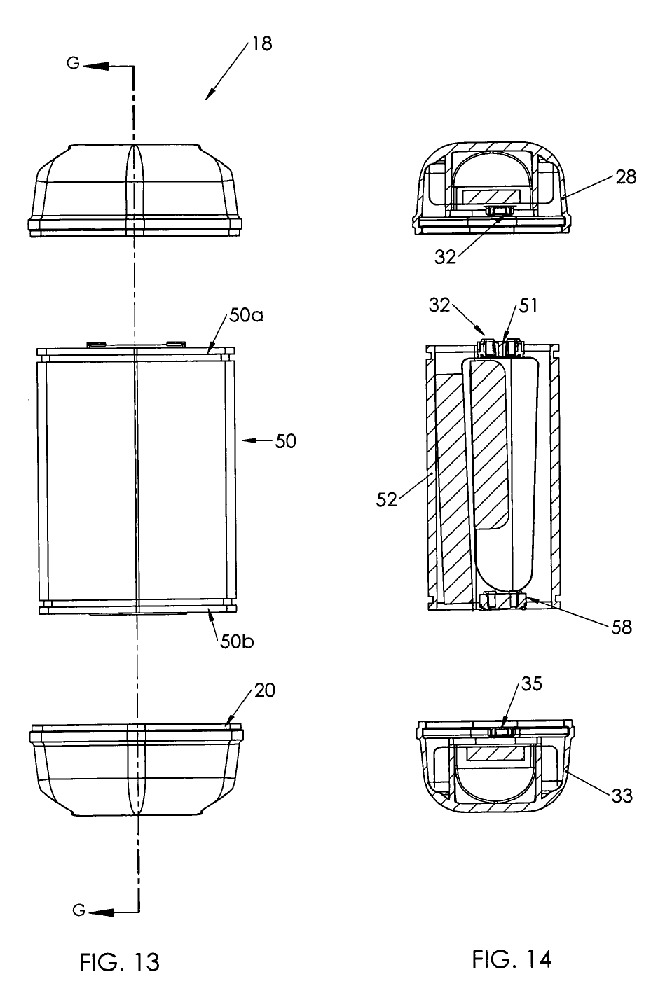

FIGS. 13 and 14 show an alternate capsule body 50 which is adapted to allow for modular caps to be interchangeably attached to both ends of the body. As shown, a pressure sensing cap 18 is attached to one end of body 50 and a pH sensing cap 20 is attached to the other end of body 50. Capsule body 50 includes the same internal components as with body 16 housed in a cylindrical shell 52. However, rather then having a single electrical receptacle 51, body 50 includes a second electrical receptacle 58 at the opposed end. Thus, electrical plug 32 of cap 18 may be inserted into receptacle 51 of capsule body 50, and plug 35 of cap 21 may be inserted into receptacle 58 of capsule body 50 to provide an ingestible capsule that senses both pH and pressure. With this embodiment, different sensing configurations may be formed by the user as desired. For example, caps having different sensors may be attached to the ends of capsule body 50 or, alternatively, caps having the same sensors may be attached to the ends of capsule body 50. As with the first embodiment, caps 18 and 20 and body 50 are connected both electrically and mechanically. Thus, shell 28 of cap 18 attaches to end 50a of shell 52 and shell 33 of cap 20 attaches to the other end 50b of shell 52. As discussed above, in this embodiment the shells are also attached or connected using a snap connection. However, other connecting methods or features may be used to provide the attachment such that the capsule does not leak when it is ingested and passes through the gastrointestinal tract of the subject.

Once the desired capsule is put together by the user, it is ingested by the subject. As the capsule passes through the gastrointestinal tract of the subject, the pH sensor and/or pressure sensor take measurements and body 16 transmits the measurements to a receiver being worn by the user, where they are stored.

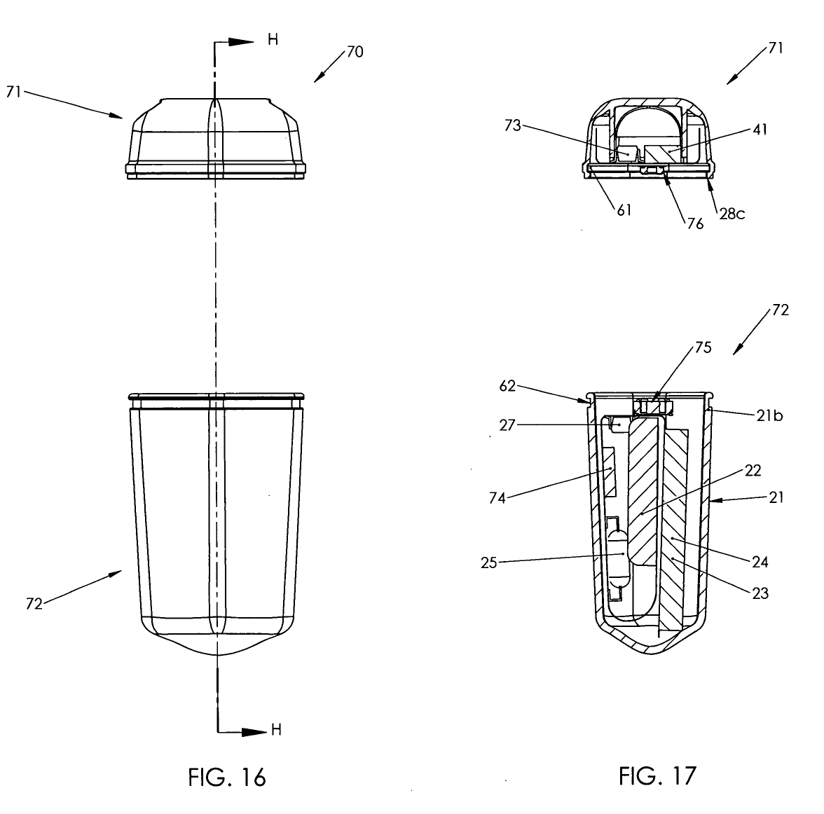

FIGS. 16-18 show a third embodiment 70 of the modular capsule. Capsule 70 is similar to the first embodiment in that capsule body 72 includes a power supply 22, a transmitter 23, an antenna 24, an activation switch 25 and a temperature sensor 27 housed in a hard shell or casing 21. However, in this embodiment capsule body 72 also houses microprocessor 74.

Cap 71 is similar to caps 18-20 in that it is attachable to body 72 and comes in different versions having different sensors or combinations of sensors for sensing parameters of the gastrointestinal tract of a subject. However, cap 71 differs in that it does not contain a microprocessor and instead contains a settable ID tag programmed into non-volatile memory 73. The settable ID tag is used to indicate the type of sensor or cap being attached to capsule body 72. For example, if cap 71 is a version that contains a pressure sensor 29 it has a first ID tag. If it is a version that contains a pH sensor it has a second and different ID tag. In this embodiment, the ID tag is programmed into an EEPROM or flash memory or set using a DIP switch during manufacturing, and the EEPROM or DIP switch is a I2C-bus compatible device which allows it to communicate directly through the I2C-bus to microprocessor 74 in capsule body 72. Thus, in this embodiment there is no need for additional interfacing. A four position Dip Switch or 4-bit EEPROM allows for 16 different versions of modular sensing cap 71 to be identified and a five position Dip Switch or 5-bit EEPROM allows for 32 different versions of modular sensing cap 71 to be identified and used with a single body 72. Once activated, the ID tag acts as a control line into processor 74. The ID tag is read and variables within the operating program are set according to a look-up table. These variables may include parameters which are unique to the particular sensor on the subject cap 71, such as the sampling rate of the sensor, the transmission burst duration and the rate of transmission bursts.

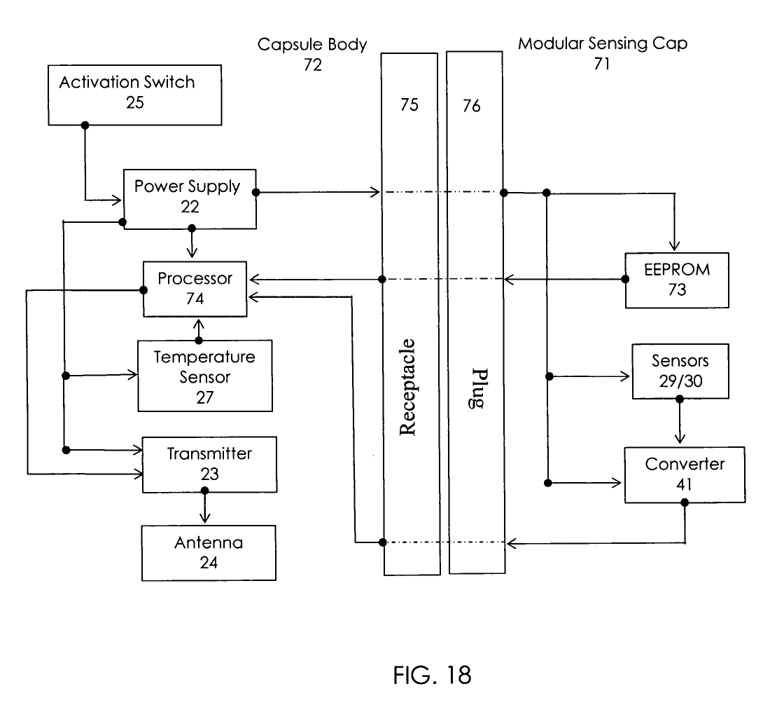

Different versions of cap 71, each version having a different sensor(s) may be attached to body 72 as in the first embodiment. As shown in FIG. 18, cap 70 includes an I-O connector having an electrical connecting input or plug 76 on the subject cap 71 and an electrical output connection, port or receptacle 75 in body 72. Power supply 22 is connected to a transmitter 23, temperature sensor 27 and processor 74 in capsule body 72, and is connected through port 75 and plug 76 to the subject sensor(s), memory 73 and converter 41 in cap 71. As with the first embodiment, power supply 22 is activated by switch 25. The subject sensors are connected through analog to digital converter 41 and, through plug 76 and port 75, to processor 74. Temperature sensor 27 in body 72 is connected directly to processor 74. Processor 74 is connected directly to transmitter 23 and transmitter 23 is in turn connected to antenna 24.

While the above embodiments have been described in relation to the gastrointestinal tract of a human, it is contemplated that the system may be used in connection with the gastrointestinal tract of other animals.

The present invention contemplates that many changes and modifications may be made. Therefore, while the presently-preferred form of the improved modular capsule system has been shown and described, and a number of alternatives discussed, persons skilled in this art will readily appreciate that various additional changes and modifications may be made without departing from the spirit of the invention, as defined and differentiated by the following claims.

What is claimed is:

1. A modular ingestible capsule comprising:
a capsule body having:
an outer shell;
a power supply;
a transmitter connected with said power supply;
an antenna connected with said transmitter;
an activator configured to activate said power supply; and
an electrical coupling element connected with said transmitter;
a first modular sensing component configured and arranged to connect to said capsule body and having:
an outer shell configured and arranged to attach to said shell of said capsule body;
a sensor for sensing a parameter of an ingestible tract; and
an electrical coupling element connected with said sensor and configured and arranged to engage said electrical coupling element of said capsule body;
whereby said first modular sensing component may be mechanically and electrically attached to said capsule body prior to ingestion of said capsule by a subject.

2. The capsule set forth in claim 1, wherein said sensor is selected from a group consisting of a pH sensor, a pressure sensor and a temperature sensor.

3. The capsule set forth in claim 1, wherein said power supply comprises a battery.

4. The capsule set forth in claim 1, wherein said transmitter is configured to transmit data to a remote receiver.

5. The capsule set forth in claim 1, wherein said capsule body further comprises a temperature sensor.

6. The capsule set forth in claim 1, wherein said electrical coupling element of said capsule body comprises a plug and said electrical coupling element of said modular sensing component comprises a receptacle configured to receive said plug.

7. The capsule set forth in claim 1, wherein said modular sensing component further comprises a processor connected with said sensor.

8. The capsule set forth in claim 7, wherein said processor is programmed to control a sampling rate of said sensor.

9. The capsule set forth in claim 7, wherein said processor is programmed to control a transmission burst duration and a rate of transmission bursts of said transmitter.

10. The capsule set forth in claim 7, wherein said processor communicates with said sensor bi-directionally.

11. The capsule set forth in claim 1, wherein said capsule body further comprises a processor connected to said electrical coupling element of said body.

12. The capsule set forth in claim 11, wherein said modular sensing component further comprises an ID tag recognizable to said processor.

13. The capsule set forth in claim 1, wherein said sensor is an analog sensor that provides an output voltage in response to stimuli.

14. The capsule set forth in claim 13, wherein said modular sensing component further comprises an analog-digital converter configured to convert an analog signal from said sensor to a digital signal.

15. The capsule set forth in claim 1, and further comprising:
a second modular sensing component configured and arranged to connect to said capsule body and having:
an outer shell configured and arranged to attach to said shell of said capsule body;
a sensor for sensing a parameter of an ingestible tract; and
an electrical coupling element connected with said sensor and configured and arranged to engage said electrical coupling element of said capsule body;
wherein said second modular sensing component may be interchangeably attached mechanically and electrically to said capsule body with said first modular sensing component.

16. The capsule set forth in claim 1, wherein said capsule body comprises a second electrical coupling element connected with said transmitter and further comprising:
a second modular sensing component configured and arranged to connect to said capsule body and having:
an outer shell configured and arranged to attach to said shell of said capsule body;
a sensor for sensing a parameter of an ingestible tract; and
an electrical coupling element connected with said sensor and configured and arranged to engage said second electrical coupling element of said capsule body;
whereby said second modular sensing component may be mechanically and electrically attached to said capsule body prior to ingestion of said capsule by a subject.

17. The capsule set forth in claim 16, wherein said sensor of said first modular sensing component is different from said sensor of said second modular sensing component.

18. The capsule set forth in claim 17, wherein said sensor of said first modular sensing component is a pH sensor and said sensor of said second modular sensing component is a pressure sensor.

19. The capsule set forth in claim 16, wherein said sensor of said first modular sensing component is the same as said sensor of said second modular sensing component.

20. The capsule set forth in claim 19, wherein said sensors are pressure sensors.

21. The capsule set forth in claim 1, wherein said transmitter and said power supply are connected directly to said electrical coupling element of said body.

22. A method of measuring parameters of the gastrointestinal tract of a subject comprising the steps of:
providing a capsule body having:
an outer shell;
a power supply;
a transmitter connected with said power supply;
an antenna connected with said transmitter;
an activator configured to activate said power supply; and
an electrical coupling element connected with said transmitter;
providing a first modular sensing component configured and arranged to connect to said capsule body and having:
an outer shell configured and arranged to attach to said shell of said capsule body;
a sensor for sensing a parameter of an ingestible tract; and
an electrical coupling element connected with said sensor and configured and arranged to engage said electrical coupling element of said capsule body;
providing a second modular sensing component configured and arranged to connect to said capsule body and having:
an outer shell configured and arranged to attach to said shell of said capsule body;
a sensor for sensing a parameter of an ingestible tract; and
an electrical coupling element connected with said sensor and configured and arranged to engage said electrical coupling element of said capsule body;
attaching one of said first or second modular sensing components to said capsule body;
having a subject ingest said capsule;
recording measurements from said sensor of said attached modular sensing component as said capsule passes through a gastrointestinal tract of said subject; and
transmitting said measurements from said transmitter to a receiver located outside of said gastrointestinal tract of said subject.

23. The method set forth in claim 22, wherein said sensor is selected from a group consisting of a pH sensor, a pressure sensor and a temperature sensor.

24. The method set forth in claim 22, wherein said power supply comprises a battery.

25. The method set forth in claim 22, wherein said capsule body further comprises a temperature sensor.

26. The method set forth in claim 22, wherein said electrical coupling element of said capsule body comprises a plug and said electrical coupling element of said first and second modular sensing components each comprise a receptacle configured to receive said plug.

27. The method set forth in claim 22, wherein said first and second modular sensing components each further comprise a processor connected with said sensor.

28. The method set forth in claim 27, wherein said processor is programmed to control a sampling rate of said sensor.

29. The method set forth in claim 27, wherein said processor is programmed to control a transmission burst duration and a rate of transmission bursts of said transmitter.

30. The method set forth in claim 22, wherein said capsule body further comprises a processor connected to said electrical coupling element of said body.

31. The method set forth in claim 30, wherein said first and second modular sensing components each further comprise different ID tags recognizable to said processor.

32. The method set forth in claim 22, wherein said sensor is an analog sensor that provides an output voltage in response to stimuli.

33. The method set forth in claim 22, wherein said first and second modular sensing components each further comprise an analog-digital converter configured to convert an analog signal from said sensor to a digital signal.

34. The method set forth in claim 22, wherein said capsule body comprises a second electrical coupling element and further comprising the step of attaching one of said first or second modular sensing components to said capsule body.

35. The method set forth in claim 22, wherein said sensor of said first modular sensing component is different from said sensor of said second modular sensing component.

36. The method set forth in claim 35, wherein said sensor of said first modular sensing component is a pH sensor and said sensor of said second modular sensing component is a pressure sensor.

37. The method set forth in claim 22, wherein said sensor of said first modular sensing component is the same as said sensor of said second modular sensing component.

38. The method set forth in claim 36, wherein said sensors are pressure sensors.
